(19) United States
(12) Patent Application Publication
(10) Pub. No.: US 2012/0015953 A1
(43) Pub. Date: Jan. 19, 2012

(54) METHODS AND COMPOSITIONS FOR THE TREATMENT OF ALLERGY

(76) Inventors: Clay Beauregard, Fort Worth, TX (US); Peter G. Klimko, Fort Worth, TX (US); John M. Yanni, Burleson, TX (US); Daniel A. Gamache, Arlington, TX (US)

(21) Appl. No.: 13/183,194

(22) Filed: Jul. 14, 2011

Related U.S. Application Data
(60) Provisional application No. 61/365,549, filed on Jul. 19, 2010.

(51) Int. Cl. A61K 31/4985; C12Q 1/28; A61P 27/14; A61K 31/335; A61K 31/519

(52) U.S. Cl. 514/250; 514/450; 514/252.17; 435/28

(57) ABSTRACT

The present invention relates to compositions comprising an antagonist of the histamine H1 receptor and a compatible antagonist of the histamine H4 receptor. The antagonist compounds are selected to prevent the H4 receptor antagonist from interfering with the H1 receptor antagonist's suppression of acute phase allergic responses in a patient. The present invention also relates to compositions comprising a mast cell stabilizer and a compatible antagonist of the histamine H4 receptor.

### CROSS-REFERENCE TO RELATED APPLICATION

This application claims priority under 35 U.S.C. §119 to U.S. Provisional Patent Application No. 61/365,549, filed Jul. 19, 2010, the entire contents of which are incorporated herein by reference.

### FIELD OF THE INVENTION

The present invention relates generally to compositions and methods for the treatment of allergy and allergy symptoms and specifically to compositions and methods using antagonists of the histamine H1 and H4 receptors.

### BACKGROUND OF THE INVENTION

Allergy is generally characterized as an immune system overreaction to the presence of foreign protein in contact with body tissues. Allergy symptoms commonly include allergic conjunctivitis (reddening of the eyes), nasal and throat irritation resulting in coughing and sneezing. More severe cases include potentially life threatening symptoms such as hypotension, breathing difficulty, asthma, and rashes. While there is no single cure for allergy, allergy symptoms can be managed with appropriate therapies.

Allergic reactions to foreign protein generally occur via two stages; an acute or early stage, and a subsequent late stage. The acute phase is mediated in part by immune cells known as mast cells. Following exposure to a foreign protein, mast cells degranulate and release histamine and immune system mediators such as cytokines into the surrounding tissue to initiate an inflammatory response. The late stage reaction is characterized by the migration of eosinophils and/or basophils, which are immune system cells, to the site of the inflamed tissue.

Antihistamines and mast cell stabilizers are two types of drugs currently used to treat allergy. Antihistamine drugs are used to interrupt the allergic effects that histamine causes after it has been released from a mast cell. Many antihistamine drugs are marketed, such as emedastine difumarate, levocabastine hydrochloride and cetirizine and generally are antagonists of one or more histamine receptor subtypes such as H1 and H4. Mast cell stabilizers prevent mast cell degranulation and/or the release of histamine and cytokines during an allergic reaction to foreign protein. Examples of drugs marketed as mast cell stabilizers include olopatadine (see U.S. Pat. No. 5,641,805 to Hayakawa et al.) and cromolyn sodium.

Combination therapies utilizing more than one allergy therapeutic are known. For example, U.S. Pat. No. 5,192,780 to York, et al discloses the use of an antihistamine and an antiallergic for treating ophthalmic allergic responses. U.S. Pat. No. 5,149,694 to Cagle, et al. discloses compositions of tobramycin and dexamethasone for the control of infection and inflammatory response.

### BRIEF SUMMARY OF THE INVENTION

The present invention provides methods and compositions for preventing or treating allergy and allergy symptoms. Such methods and compositions may be used to prevent or treat allergy and allergy symptoms involving tissues of the eye, nose, skin, ear, gastrointestinal tract, airways or lung. The methods and compositions may also be used to treat manifestations of systemic mastocytosis. The methods of the present invention comprise topically or systemically administering to a patient a composition comprising a H4 receptor antagonist that is compatible with a co-administered H1 receptor antagonist such as olopatadine and/or a mast cell stabilizer such as cromolyn sodium.

The present inventors have found that certain H4 receptor antagonists attenuate the acute phase inhibitory activity of H1 receptor antagonists such as olopatadine. However, other H4 receptor antagonists do not cause this attenuation to a significant extent, and are considered compatible compounds for concomitant administration with H1 receptor antagonists.

The present invention also comprises techniques for identifying H4 receptor antagonists compatible with H1 receptor antagonists for concomitant administration.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a graph showing the acute phase effects of an H4 antagonist (Compound 1) administered once daily with concomitant dosing of 0.2% olopatadine in passively sensitized guinea pigs;

FIG. 2 is a graph showing the late phase effects of an H4 antagonist (Compound 1) administered once daily with concomitant dosing of 0.2% olopatadine in passively sensitized guinea pigs;

FIG. 3 is a bar graph showing the effects of different dosing regimens on the acute phase effects of an H4 antagonist (Compound 1) and 0.2% olopatadine;

FIG. 4 is a graph showing the acute phase effects of an H4 antagonist (Compound 2) administered once daily with concomitant dosing of 0.2% olopatadine in passively sensitized guinea pigs;

FIG. 5 is a graph showing the late phase effects of an H4 antagonist (Compound 2) administered once daily with concomitant dosing of 0.2% olopatadine in passively sensitized guinea pigs; and

FIG. 6 is a bar graph showing the effects of different dosing regimens on the acute phase effects of an H4 antagonist (Compound 2) and 0.2% olopatadine.

### DETAILED DESCRIPTION OF THE INVENTION

The compositions of the present invention comprise a H1 receptor antagonist and a compatible H4 receptor antagonist. In certain embodiments, the compositions of the present invention comprise a mast cell stabilizer compound and a compatible H4 receptor antagonist. As used herein, a compatible H4 receptor antagonist is one that does not cause significant attenuation of the H1 receptor antagonist's effect on the acute phase allergic response. Pharmaceutically-acceptable salts of the compounds set forth herein are also specifically contemplated.

Compositions of the present invention are useful for preventing and treating ophthalmic allergic disorders, including allergic conjunctivitis, vernal conjunctivitis, vernal keratoconjunctivitis, and giant papillary conjunctivitis; nasal allergic disorders, including allergic rhinitis and sinusitis; otic allergic disorders, including eustachian tube itching; allergic disorders of the upper and lower airways, including intrinsic and extrinsic asthma; allergic disorders of the skin, including dermatitis, eczema and urticaria; allergic disorders of the gastrointestinal tract, including systemic anaphylaxis resulting from ingestion of allergen and iatrogenic anaphylaxis caused by contrast agents used during diagnostic imaging procedures; manifestations of systemic mastocytosis including hypotension; dry eye, glaucoma, glaucomatous retinopathy, diabetic retinopathy, retinal ganglion degeneration, ocular ischemia, retinitis, retinopathies, uveitis, ocular photophobia, and of inflammation and pain associated with acute injury to the eye tissue. The compounds can also be used to treat post-operative inflammation or pain as from ophthalmic surgery such as cataract surgery and refractive surgery.

H1 receptor antagonists of the present invention include known selective and non-selective H1 receptor antagonists known to those of skill in the art. Such antagonists include, but are not limited to: emedastine, levocabastine, mequitazine, chlorpheniramine, brompheniramine, astemizole, cetirizine, terfenadine, rocastine, loratadine, 5-[2-[4-bis(4-fluorophenyl)hydroxymethyl-1-piperidinyl]ethyl]-3-methyl]-2-oxazolidinone ethanedioate) pyrilamine, clemastine, azelastine, ketotifen, olopatadine, and mapinastine.

Mast cell stabilizers suitable for use in combination with H4 antagonists of the present invention include but are not limited to cromolyn sodium, pemirolast potassium, repirinast, suplatast tosylate, amlexanox, oxatomide, acitazanolast, olopatadine, and lodoxamide tromethamine.

In a preferred composition of the present invention, olopatadine is used as the selected H1 receptor antagonist. Olopatadine is (Z)-11-(3-dimethylamino propylidene)-6,11-dihydrodibenz[b,e]-oxepin-2-acetic acid. Olopatadine can be made using the methods disclosed in U.S. Pat. No. 5,116,863 to Oshima et al., the entire contents of which are hereby incorporated by reference. A preferred pharmaceutically-acceptable salt is olopatadine hydrochloride. In yet another preferred composition of the present invention, cromolyn sodium (a mast cell stabilizer) is combined with a compatible H4 antagonist of the present invention.

The compositions of the present invention also comprise a selective or non-selective H4 receptor antagonist. Such H4 receptor antagonists include, but are not limited to, those disclosed in WO/2010/030785 to Borchardt et al., and in U.S. Provisional Patent Application Ser. Nos. 61/312,615 and 61/312,619 the entire contents of which are hereby incorporated by reference. Preferred H4 receptor antagonists of the present invention are of the following Formula (I):

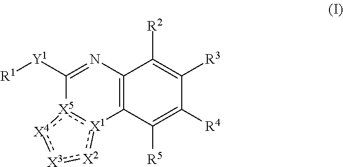

where the ring comprising X¹-X⁵ is aromatic;

X¹ and X⁵ are independently selected from the group consisting of C, CH and N;

X² is selected from the group consisting of [C(R⁶)(R⁷)]ₙ, NR⁸, O and S;

X³ is selected from the group consisting of)[C(R⁹)(R¹⁰]ₘ, NR¹¹, O, and S;

X⁴ is selected from the group consisting of [C(R¹²)(R¹³)], NR¹⁴, O and S;

n and m are each an integer from 1 to 2;

Y¹ is selected from the group consisting of a bond, lower alkyl, lower alkoxy, OR¹⁵, NR¹⁶R¹⁷, and lower aminoalkyl;

R¹ is selected from the group consisting of:

null, when Y¹ is selected from the group consisting of OR¹⁵, and NR¹⁶R¹⁷; and

aryl, heterocycloalkyl, cycloalkyl, and heteroaryl, any of which may be optionally substituted, when Y¹ is a bond;

R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;

R⁶, R⁷, R⁹, R¹⁰, R¹², and R¹³ are independently selected from the group consisting of null, hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;

R⁸, R¹¹, and R¹⁴ are independently selected from the group consisting of null, hydrogen, alkyl, heteroalkyl, alkoxy, haloalkyl, perhaloalkyl, aminoalkyl, C-amido, carboxyl, acyl, hydroxy, aryl, arylalkyl, cycloalkyl, cycloalkylalkyl, heterocycloalkyl, heterocycloalkylalkyl, heteroaryl, and heteroarylalkyl, any of which may be optionally substituted;

R¹⁵ and R¹⁶ are independently selected from the group consisting of aminoalkyl, alkylaminoalkyl, aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted; and

R¹⁷ is independently selected from the group consisting of hydrogen, aminoalkyl, alkylaminoalkyl aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted;

with the proviso that the following two compounds are excluded:
4-(piperazin-1-yl)-8-(trifluoromethyl)-[1,2,4]triazolo[4,3-a]quinoxaline and 4-(4-methylpiperazin-1-yl)-8-(trifluoromethyl)-[1,2,4]triazolo[4,3-a]quinoxaline.

An especially preferred H4 receptor antagonist of the present invention is Compound 2, shown below:

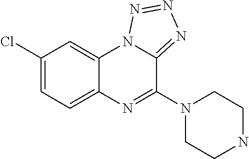

### Compound 2 (8-chloro-4-(piperazin-1-yl)tetrazolo[1,5-a]quinoxaline)

Other especially preferred H4 antagonists of the present invention include the following:

| Structure | Name |
|---|---|
| 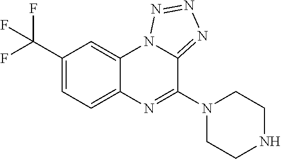 | 4-(piperazin-1-yl)-8- (trifluoromethyl)tetrazolo [1,5-a]quinoxaline |
| 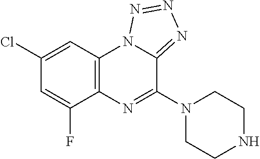 | 8-chloro-6-fluoro-4-(piperazin- 1-yl)tetrazolo[1,5-a)quinoxaline |
| 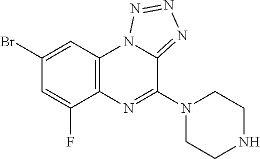 | 8-bromo-6-fluoro-4-(piperazin-1- yl)tetrazolo[1,5-a]quinoxaline |
| 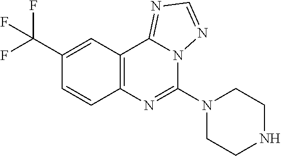 | 5-(piperazin-1-yl)-9-(trifluoromethyl)- [1,2,4]triazolo[1,5-c]quinazoline |
| 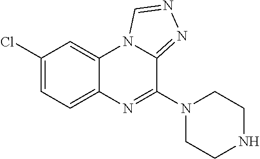 | 8-chloro-4-(piperazin-1-yl)- [1,2,4]triazolo[4,3-a]quinoxaline |
| 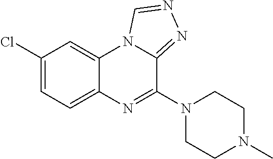 | 8-chloro-4-(4-methylpiperazin-1-yl)- [1,2,4]triazolo[4,3-a]quinoxaline |
| 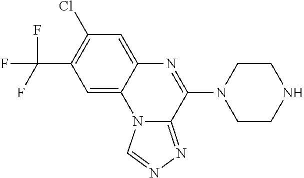 | 7-chloro-4-(piperazin-1-yl)-8- (trifluoromethyl)-[1,2,4]triazolo [4,3-a]quinoxaline |
| 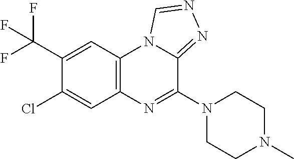 | 7-chloro-4-(4-methylpiperazin-1-yl)-8- (trifluoromethyl)-[1,2,4]triazolo [4,3-a]quinoxaline |

The concentration of the H1 receptor antagonist and the H4 receptor antagonist in the compositions of the present invention range from about 0.0001 to 5% (w/v) measured separately and not in aggregate, preferably from about 0.001 to 0.25% (w/v), and most preferably from about 0.1 to 0.25% (w/v).

In addition to a H1 receptor antagonist and a compatible H4 receptor antagonist, the compositions of the present invention optionally comprise one or more additional components. Such components include, but are not limited to, tonicity agents, preservatives, chelating agents, buffering agents, surfactants, co-solvents, and antioxidants. Other components used in certain embodiments are solubilizing agents, stabilizing agents, comfort-enhancing agents, polymers, emollients, pH-adjusting agents and/or lubricants. Components that may be used in certain compositions of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, and mixtures of those products.

Surfactants utilized in the compositions of the present invention can be cationic, anionic, nonionic or amphoteric. Preferred surfactants are neutral or noninonic surfactants which may present in amounts up to 5 w/v %. Surfactants that may be used with certain embodiments of the present invention include, but are not limited to, polyethylene glycol ethers or esters of fatty acids, polyoxyethylene-polyoxypropylene block copolymers of ethylene diamine (e.g., poloxamines such as Tetronic 1304 or 1107), polyoxypropylene-polyoxyethylene glycol nonionic block copolymers (e.g., poloxamers, such as Pluronic F-127), and p-isooctylpolyethylen phenol formaldehyde polymers (e.g., Tyloxapol).

In certain embodiments of the present invention, suitable cosolvents include glycerin, propylene glycol and polyethylene glycol.

Buffering agents which may be incorporated into compositions of the present invention include, but are not limited to, alkaline metal salts, such as potassium or sodium carbonates, acetates, borates, phosphates and citrates, and weak acids, such as acetic acids and boric acids. The preferred buffering agents are alkaline metal borates, such as sodium or potassium borates. Other pH-adjusting agents, such as inorganic acids and bases, may also be utilized. For example, hydrochloric acid or sodium hydroxide may be employed in concentrations suitable for ophthalmic compositions. The above-described buffering agents are generally present in amounts from about 0.1 to about 2.5 w/v %, preferably from about 0.5 to about 1.5% w/v %.

The compositions of the present invention are preferably isotonic, or slightly hypotonic, and generally have an osmolality in the range of 210-320 mOsm/kg, and preferably have an osmolality in the range of 235-300 mOsm/kg. This may require a tonicity agent to bring the osmolality of the formulation to the desired level. Tonicity-adjusting agents include, but are not limited to, sodium chloride, glycerin, sorbitol, or mannitol.

The compositions set forth herein may comprise one or more preservatives. Examples of preservatives include p-hydroxybenzoic acid ester, alkyl-mercury salts of thiosalicylic acid, such as, for example, thiomersal, phenylmercuric nitrate, phenylmercuric acetate or phenylmercuric borate, quaternary ammonium compounds such as, for example, polyquaternium-1, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, polyhexamethylene biguanide, sodium perborate, or sorbic acid. In preferred embodiments, the formulation may be self-preserved that no preservation agent is required.

The pH of the compositions of the present invention is generally in the physiologically-compatible range of 3.0 to 8.0. Preferred ophthalmic compositions have a pH of between 6.0 and 8.0.

In particular embodiments, compositions of the present invention are suitable for topical application to mammalian eyes. For example, for ophthalmic administration, the formulation may be a solution, a suspension, a gel, water-in-oil and oil-in-water emulsions, or an ointment. Preferred compositions for ophthalmic administration will be aqueous solution in the form of drops. The term “aqueous” typically denotes an aqueous formulation wherein the excipient is >50%, more preferably >75% and in particular >90% by weight water. These drops may be delivered from a single dose ampoule which may preferably be sterile and thus render bacteriostatic components of the formulation unnecessary. Alternatively, the drops may be delivered from a multi-dose bottle which may preferably comprise a device which extracts preservative from the formulation as it is delivered, such devices being known in the art.

Compositions of the present invention may be administered topically (i.e., local, organ-specific delivery) or systemically by means of conventional topical or systemic compositions, such as solutions, suspensions or gels for the eye and ear; nasal sprays or mists for the nose; metered dose inhalers for the lung; solutions, gels, creams or lotions for the skin; oral dosage forms including tablets or syrups for the gastrointestinal tract; and parenteral dosage forms including injectable compositions. The concentration of the H1 and H4 receptor antagonists in the compositions of the present invention will depend on the selected route of administration and dosage form.

In a method according to the present invention, an H1 receptor antagonist is administered concomitantly with a compatible H4 receptor antagonist in either the same composition or in separate compositions administered contemporaneously. In a preferred embodiment, the H1 receptor antagonist and compatible H4 receptor antagonist are administered within an hour of each other.

In particular embodiments, a composition of the present invention is administered once a day. However, the compositions of the present invention may also be formulated for administration at any frequency of administration, including once a week, once every 5 days, once every 3 days, once every 2 days, twice a day, three times a day, four times a day, five times a day, six times a day, eight times a day, every hour, or any greater frequency. Such dosing frequency is also maintained for a varying duration of time depending on the therapeutic regimen. The duration of a particular therapeutic regimen may vary from one-time dosing to a regimen that extends for months or years. One of ordinary skill in the art would be familiar with determining a therapeutic regimen for a specific indication. Factors involved in this determination include the disease to be treated, particular characteristics of the subject, and the particular composition.

### EXAMPLES

The following examples are presented to further illustrate selected embodiments of the present invention.

### Example 1

| Ingredient | % w/v |
|---|---|
| Compound 2 | 0.2 |
| Olopatadine | 0.2 |
| HP-Guar | 0.15 |
| Boric Acid | 0.35 |
| Sodium Borate | 0.11 |
| Sodium Chloride | 0.7 |
| Sodium Chlorite | 0.006 |
| Sodium Hydroxide/Hydrochloric Acid | pH adjust to 7.0 |

### Example 2

H1 and H4 antagonists were evaluated using an in vivo assay to determine the effect of the H4 antagonist on a co-administered H1 antagonist. Male Hartley VAF outbred guinea pigs were passively sensitized to ovalbumin by a single OD subconjunctival injection of undiluted guinea pig anti-ovalbumin antiserum 24 hours before OD topical challenge with 500 μg ovalbumin in saline. Control animals were injected with saline only and challenged with ovalbumin. To determine acute phase drug efficacy, 30 min after challenge animals were clinically scored by a masked observer for severity of signs of conjunctivitis based on a standard scale.

Test compounds were administered topically 1 hour prior to challenge. Twenty-four hours after challenge, animals were euthanized and conjunctivae were harvested for determination of tissue eosinophil peroxidase (EPO) concentration as a marker of allergic inflammation and late phase effects. Homogenates of freshly collected tissues were prepared by shaking the tissues in 2 mL round-bottom tubes containing 0.5 mL of homogenization buffer (50 mM Tris HCl, pH 8.0, 6 mM KBr) and one 5-mm stainless steel bead on a Qiagen TissueLyser at 30 Hz for 5 min. Homogenates were frozen and thawed once, then centrifuged at 10,000 rpm for 5 min. EPO activity in supernatants was measured by reacting diluted homogenates with a solution of 6 mM o-phenylenediamine substrate and 8.8 mM H₂O₂ in homogenization buffer for 3 min. The reaction was stopped with 4M H₂SO4 and absorbances were measured at 490 nM on a spectrophotometry plate reader. Total EPO in samples was calculated from a standard curve of recombinant human EPO in each assay. EPO activity was normalized to total protein concentration (Pierce BCA assay) in supernatants. Background EPO activity was determined from the unsensitized, antigen-challenged control group. Percent inhibition was calculated from the sensitized, antigen-challenged, vehicle-treated control group in each experiment. Ovalbumin-injected animals dosed topically with 0.1% w/v dexamethasone (dex) served as positive control. Groups were compared by ANOVA with Dunnett's or Tukey's post-hoc tests where appropriate with significance assigned at the 95% confidence level.

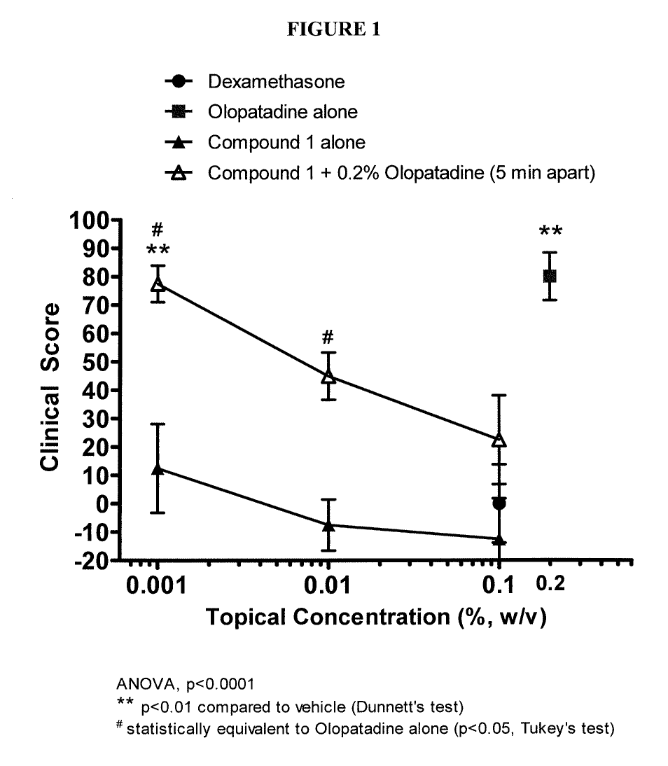

FIG. 1 is a graph showing the acute phase effects of an H4 antagonist (Compound 1,4-(4-methylpiperazin-1-yl)-8-(trifluoromethyl)-[1,2,4]triazolo[4,3-a]quinoxaline) administered once daily with concomitant dosing of 0.2% olopatadine, an antagonist of H1 that also has mast cell stabilization activity. Compound 1 was dosed first, followed by olopatadine 5 minutes later. Antigen challenge was 60 minutes after the compound 1 dose. FIG. 1 demonstrates that Compound 1 has a dose dependent effect on the inhibition of the acute clinical score relative to olopatadine alone.

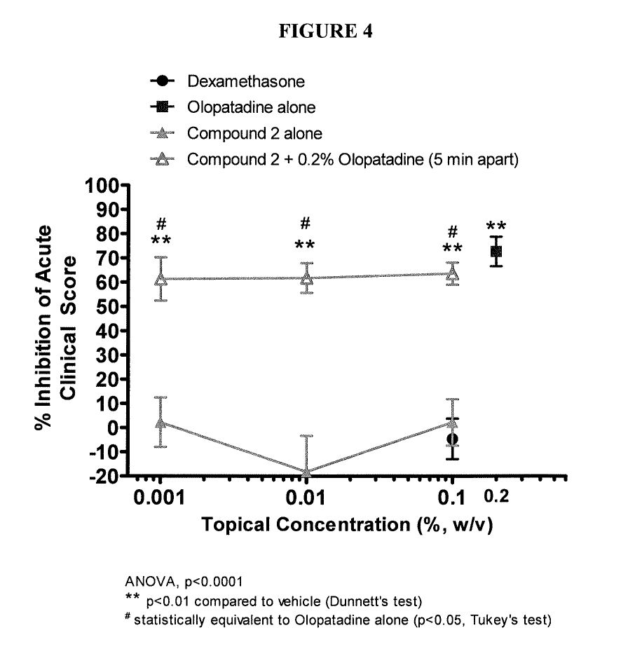

FIG. 4 is a graph showing the acute phase effects of an H4 antagonist (Compound 2) administered once daily with concomitant dosing of 0.2% olopatadine as in the Compound 1 experiment. Unlike Compound 1, Compound 2 does not show suppression of olopatadine's acute phase effects. Neither Compound 1 nor Compound 2 demonstrate any effect on acute phase clinical score on their own.

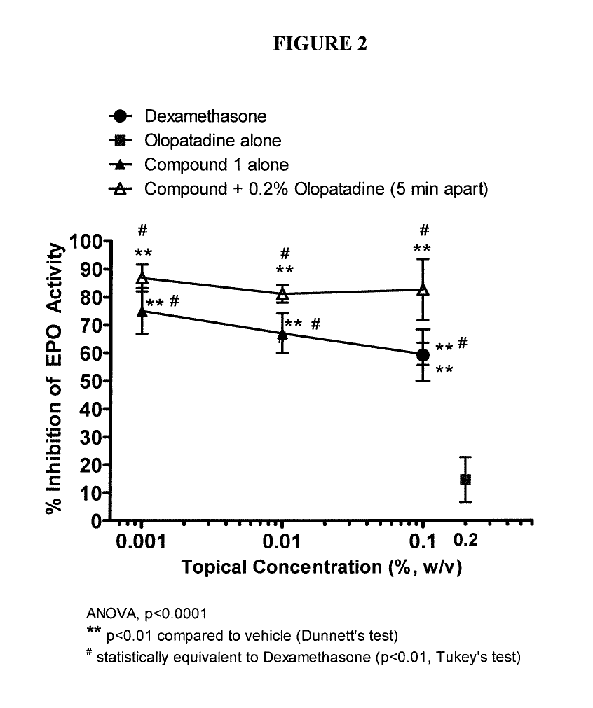

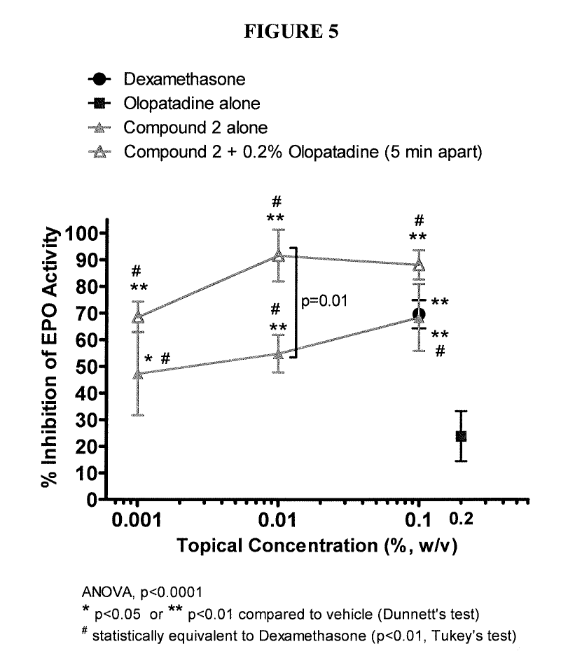

FIGS. 2 and 5 show that, respectively, Compound 1 and Compound 2 are very good inhibitors of late phase allergic response. Concomitant dosing with olopatadine causes a slight increase in the inhibition of late phase response by both Compound 1 and Compound 2.

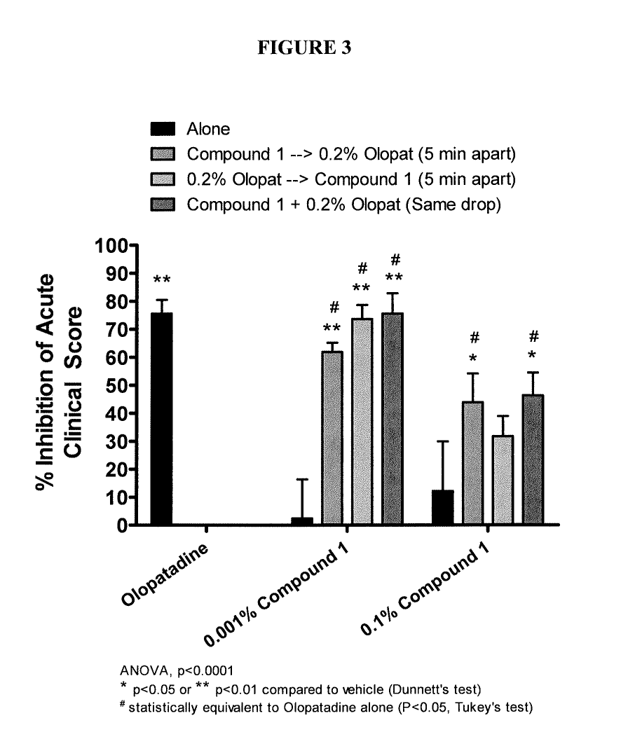

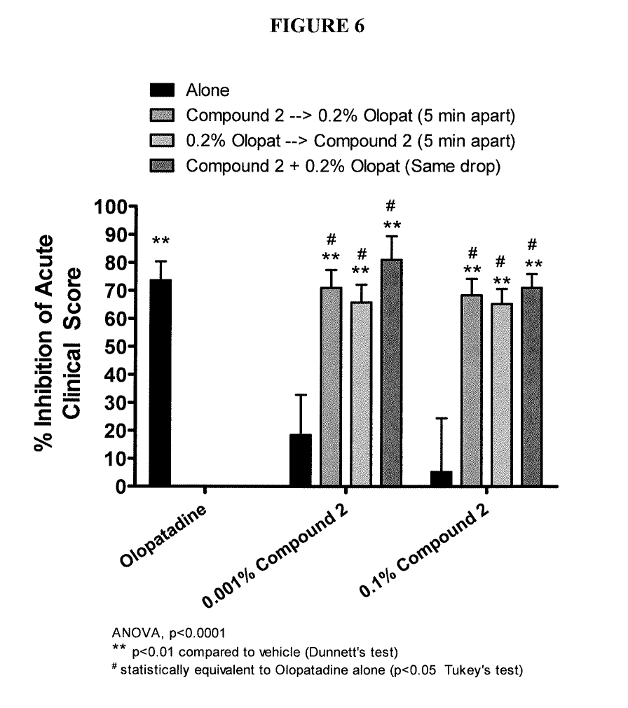

FIGS. 3 and 6 are bar graphs confirming the reduction of olopatadine's acute phase effects and dose dependency when Compound 1 is concomitantly administered (before, after, or in the same drop as olopatadine). These effects on acute phase response are not seen with concomitant administration of Compound 2 and olopatadine.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A sterile, aqueous ophthalmic composition comprising a histamine H1 receptor antagonist and a compatible histamine H4 receptor antagonist.

2. A composition of claim 1 wherein the H1 receptor antagonist is olopatadine.

3. A composition of claim 2 wherein the olopatadine concentration is from 0.0001 to 5% (w/v).

4. A composition of claim 2 wherein the olopatadine concentration is from 0.1 to 0.25% (w/v).

5. A composition of claim 1 wherein the H4 receptor antagonist is of Formula (I): 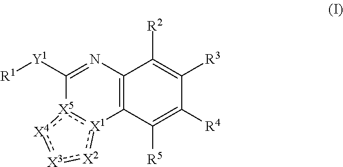
where the ring comprising X¹-X⁵ is aromatic;
X¹ and X⁵ are independently selected from the group consisting of C, CH and N;
X² is selected from the group consisting of [C(R⁶)(R⁷)]ₙ, NR⁸, O and S;
X³ is selected from the group consisting of [C(R⁹)(R)]ₘ, NR¹¹, O, and S;
X⁴ is selected from the group consisting of [C(R¹²)(R¹³)], NR¹⁴, O and S;
n and m are each an integer from 1 to 2;
Y¹ is selected from the group consisting of a bond, lower alkyl, lower alkoxy, OR¹⁵, NR¹⁶R¹⁷, and lower aminoalkyl;
R¹ is selected from the group consisting of:
null, when Y¹ is selected from the group consisting of OR¹⁵, and NR¹⁶R¹⁷; and
aryl, heterocycloalkyl, cycloalkyl, and heteroaryl, any of which may be optionally substituted, when Y¹ is a bond;
R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
R⁶, R⁷, R⁹, R¹⁰, R¹², and R¹³ are independently selected from the group consisting of null, hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
R⁸, R¹¹, and R¹⁴ are independently selected from the group consisting of null, hydrogen, alkyl, heteroalkyl, alkoxy, haloalkyl, perhaloalkyl, aminoalkyl, C-amido, carboxyl, acyl, hydroxy, aryl, arylalkyl, cycloalkyl, cycloalkylalkyl, heterocycloalkyl, heterocycloalkylalkyl, heteroaryl, and heteroarylalkyl, any of which may be optionally substituted;
R¹⁵ and R¹⁶ are independently selected from the group consisting of aminoalkyl, alkylaminoalkyl, aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted; and
R¹⁷ is independently selected from the group consisting of hydrogen, aminoalkyl, alkylaminoalkyl aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted;
with the proviso that the following two compounds are excluded:
4-(piperazin-1-yl)-8-(trifluoromethyl)-[1,2,4]triazolo[4,3-a]quinoxaline and 4-(4-methylpiperazin-1-yl)-8-(trifluoromethyl)-[1,2,4]triazolo[4,3-a]quinoxaline.

6. A composition of claim 1 wherein the H4 receptor antagonist is selected from the group consisting of the following compounds:

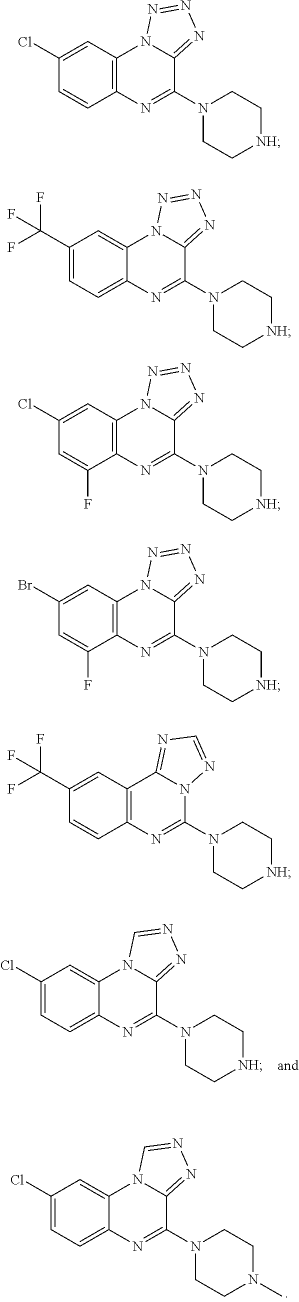

7. A composition of claim 5 wherein the H4 receptor antagonist concentration is from 0.0001 to 5% (w/v).

8. A composition of claim 5 wherein the H4 receptor antagonist concentration is from 0.1 to 0.25% (w/v).

9. A composition of claim 6 wherein the Compound 2 concentration is from 0.0001 to 5% (w/v).

10. A composition of claim 6 wherein the Compound 2 concentration is from 0.1 to 0.25% (w/v).

11. A method for treating or controlling allergic disorders in patients comprising topically administering a composition comprising a histamine H1 receptor antagonist and a compatible histamine H4 receptor antagonist.

12. A method according to claim 11 wherein the H1 receptor antagonist is olopatadine.

13. A method according to claim 11 wherein the H4 receptor antagonist is of Formula (I): 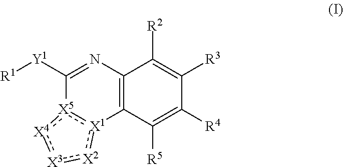
where the ring comprising X¹-X⁵ is aromatic;
X¹ and X⁵ are independently selected from the group consisting of C, CH and N;
X² is selected from the group consisting of [C(R⁶)(R⁷)]ₙ, NR^{B}, O and S;
X³ is selected from the group consisting of [C(R⁹)(R¹⁰)]ₘ, NR¹¹, O, and S;
X⁴ is selected from the group consisting of [C(R¹²)(R¹³)], NR¹⁴, O and S;
n and m are each an integer from 1 to 2;
Y¹ is selected from the group consisting of a bond, lower alkyl, lower alkoxy, OR¹⁵, NR¹⁶R¹⁷, and lower aminoalkyl;
R¹ is selected from the group consisting of:
null, when Y¹ is selected from the group consisting of OR¹⁵, and NR¹⁶R¹⁷; and
aryl, heterocycloalkyl, cycloalkyl, and heteroaryl, any of which may be optionally substituted, when Y¹ is a bond;
R², R³, R⁴, and R⁵ are independently selected from the group consisting of hydrogen, alkyl, alkenyl, heteroalkyl, alkoxy, halogen, haloalkyl, perhaloalkyl, perhaloalkoxy, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
R⁶, R⁷, R⁹, R¹⁰, R¹², and R¹³ are independently selected from the group consisting of null, hydrogen, alkyl, heteroalkyl, alkoxy, halogen, haloalkyl, perhaloalkyl, amino, aminoalkyl, amido, carboxyl, acyl, hydroxy, cyano, nitro, aryl, arylalkyl, cycloalkyl, cycloalkylalkyl, heterocycloalkyl, heterocycloalkylalkyl, heteroaryl, and heteroarylalkyl, any of which may be optionally substituted;
R⁸, R¹¹, and R¹⁴ are independently selected from the group consisting of null, hydrogen, alkyl, heteroalkyl, alkoxy, haloalkyl, perhaloalkyl, aminoalkyl, C-amido, carboxyl, acyl, hydroxy, aryl, arylalkyl, cycloalkyl, cycloalkylalkyl, heterocycloalkyl, heterocycloalkylalkyl, heteroaryl, and heteroarylalkyl, any of which may be optionally substituted;
R¹⁵ and R¹⁶ are independently selected from the group consisting of aminoalkyl, alkylaminoalkyl, aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted; and
R¹⁷ is independently selected from the group consisting of hydrogen, aminoalkyl, alkylaminoalkyl aryl, arylalkyl, cycloalkyl, cycloalkylalkyl, ether, heterocycloalkyl, lower alkylaminoheterocycloalkyl, heterocycloalkylalkyl, heteroaryl, and heteroarylalkyl, any of which may be optionally substituted;
with the proviso that the following two compounds are excluded:
4-(piperazin-1-yl)-8-(trifluoromethyl)-[1,2,4]triazolo[4,3-a]quinoxaline and 4-(4-methylpiperazin-1-yl)-8-(trifluoromethyl)-[1,2,4]triazolo[4,3-a]quinoxaline.

14. A method according to claim 11 wherein the H4 receptor antagonist is selected from the group consisting of the following compounds:

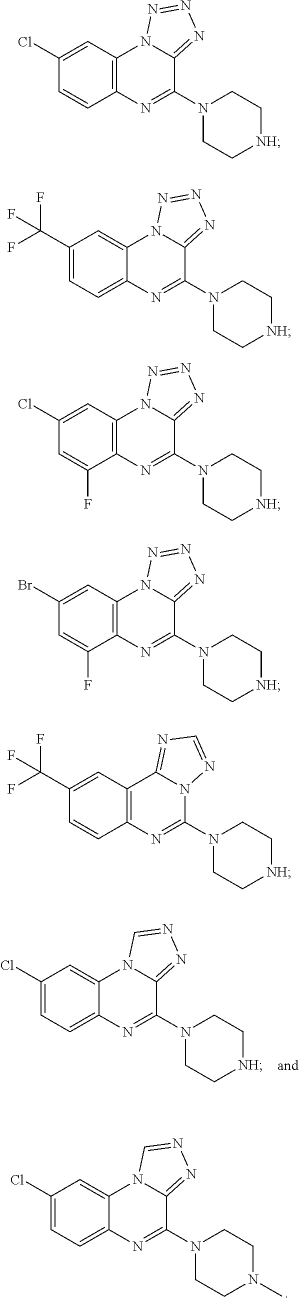

15. A method according to claim 11 wherein said allergic disorders are ophthalmic or nasal allergic disorders.

16. A method for selecting compatible histamine H1 receptor antagonists and histamine H4 receptor antagonist for concomitant administration comprising:
comparing the allergic acute phase effects of the H1 receptor antagonist alone and with a H4 receptor antagonist; and
selecting the compatible H1 receptor antagonist and the H4 receptor antagonist if said comparing shows no suppression of allergic acute phase effects.
